(19) United States
(12) Patent Application Publication
(10) Pub. No.: US 2022/0409146 A1
(43) Pub. Date: Dec. 29, 2022

(54) STATIONARY X-RAY SOURCE ARRAY FOR DIGITAL TOMOSYNTHESIS

(71) Applicant: CARESTREAM HEALTH, INC., Rochester, NY (US)

(72) Inventors: Xiaohui WANG, Pittsford, NY (US); David H. FOOS, Webster, NY (US); Levon O. VOGELSANG, Webster, NY (US)

(21) Appl. No.: 17/846,057

(22) Filed: Jun. 22, 2022

Related U.S. Application Data
(60) Provisional application No. 63/213,784, filed on Jun. 23, 2021.

(51) Int. Cl. A61B 6/02; A61B 6/00; G06T 7/00

(52) U.S. Cl. CPC A61B 6/025; A61B 6/5235; G06T 7/0012; A61B 6/503; G06T 2207/10112; G06T 2207/10116; G06T 2207/30048; G06T 2207/30061

(57) ABSTRACT

A plurality of radiographic images are captured of a portion of a patient in periodic motion, such as cardiac images (heartbeat motion) or lungs (breathing motion). A first subset of the captured radiographic images are identified as having a common first capture time relative to a phase of the periodic motion. A first 3D image is reconstructed using the first subset of captured radiographic images. Additional subsets of the radiographic images are processed similarly based on their common capture time relative to the phase of the periodic motion.

### CROSS REFERENCE TO RELATED APPLICATIONS

This application claims priority to U.S. Patent Application Ser. No. 63/213,784, filed Jun. 23, 2021, in the name of Wang et al., and entitled STATIONARY X-RAY SOURCE ARRAY FOR DIGITAL TOMOSYNTHESSIS, which is hereby incorporated by reference herein in its entirety.

This application is related in certain respects to International Application No. PCT/US2022/030804, filed May 25, 2022, in the name of Foos et al., and entitled CARDIAC GATED DIGITAL TOMOSYNTHESIS which is hereby incorporated by reference herein in its entirety.

### BACKGROUND OF THE INVENTION

The subject matter disclosed herein relates to digital tomosynthesis (DT) imaging using a stationary x-ray source array.

Patient motion during a digital tomosynthesis scan introduces blur artifacts into the captured images. In addition, the reconstructed DT volume is static, which does not reflect the dynamic nature of the patient anatomy in motion, such as lung breathing or cardiac motion.

The discussion above is merely provided for general background information and is not intended to be used as an aid in determining the scope of the claimed subject matter.

### BRIEF DESCRIPTION OF THE INVENTION

The invention solves the above problems by timing the projection image acquisition at different phases of periodic anatomy motion, wherein the acquisition occurs through multiple cycles of the periodic motion. The projections captured at the same phase of motion are used for reconstruction, and a different reconstruction is generated for each captured phase of the periodic motion. A plurality of radiographic images of a patient may be captured, grouped and reconstructed according to a common periodic phase depicted in each of the captured image groups. The 3D reconstruction is performed for each phase group. A 4D (3D+time) motion volume may be generated and displayed using the plurality of 3D reconstructions for anatomical motion assessment.

A plurality of radiographic images are captured of a portion of a patient in periodic motion, such as cardiac images (heartbeat motion) or lungs (breathing motion). A first subset of the captured radiographic images are identified as having a common first capture time relative to a phase of the periodic motion. A first 3D image is reconstructed using the first subset of captured radiographic images. Additional subsets of the radiographic images are processed similarly based on their common capture time relative to the phase of the periodic motion. An advantage that may be realized in the practice of some disclosed embodiments of the present invention is a sharper 3D reconstruction and the ability to display the sharper 3D reconstructions in motion (4D).

In one embodiment, a plurality of radiographic images of a portion of a patient are captured, wherein the portion of the patient is in periodic motion. A first subset of the captured radiographic images are identified and are used for reconstructing a 3D (volume) image. The first subset is identified based on each image being captured at a same instant relative to a phase of the periodic motion of the portion of the patient.

In another embodiment, a plurality of radiographic heart or lung images of a patient are captured, wherein the heart or lungs of the patient are in periodic motion. A first subset of the captured radiographic heart or lung images are selected according to their common capture time relative to a phase of the periodic motion. A 3D (volume) image is reconstructed using the first subset of the captured radiographic heart or lung images.

The summary descriptions above are not meant to describe individual separate embodiments whose elements are not interchangeable. In fact, many of the elements described as related to a particular embodiment can be used together with, and possibly interchanged with, elements of other described embodiments. Many changes and modifications may be made within the scope of the present invention without departing from the spirit thereof, and the invention includes all such modifications.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings below are intended to be drawn neither to any precise scale with respect to relative size, angular relationship, relative position, or timing relationship, nor to any combinational relationship with respect to interchangeability, substitution, or representation of a required implementation, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a schematic diagram of a method of capturing a plurality of tomosynthesis images of a patient;

FIG. 2 is a schematic diagram illustrating a method of capturing radiographic images of a portion of a patient in motion; and

FIG. 3 illustrates instances of image capture at common phases along multiple cycles of patient motion.

### DETAILED DESCRIPTION OF THE INVENTION

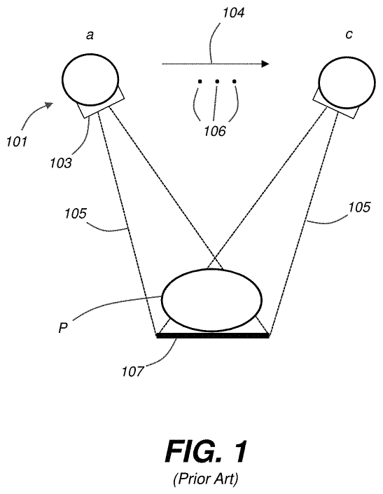

The schematic diagram of FIG. 1 shows an example tomosynthesis system using a movable tube head 101 having a collimator 103, wherein the tube head 101 is moved from a starting position a to a terminal position c while exposing a patient P multiple times during movement therebetween using x-ray beams 105 emitted by an x-ray source in the tube head 101 to capture corresponding multiple radiographic images of the patient P using a portable, stationary, digital radiographic (DR) panel detector 107 positioned under, or behind, patient P. In one embodiment, a single pass of the tube head 101 from initial position a to terminal position c may have a time duration sufficient for K full cycles of periodic cardiac or respiratory motion to occur in a portion of the anatomy of patient P, while N projection images of patient P are captured, where K and N are preselected integer values. Thus, the number of desired radiographic images of the patient P anatomy and the measured time duration per period of cardiac or respiratory motion will determine timing and frequency of the x-ray source activation along the imaging path 104 from a to c. As the tube head 101 is moved along the imaging path 104, the x-ray source therewithin is selectively activated to emit x-ray beams 105. The patient P may be positioned to lie flat over the detector 107 or the patient P may be standing adjacent the detector 107 which may be mounted on a vertical wall unit. The portable DR detector 107 is positioned behind the patient with respect to the tube head 101. In one embodiment, there is no inherent mechanical linkage between x-ray tube head 101 and the DR detector 107. The tube head 101 may be moved to several imaging positions 106, or angles, relative to the patient P and the portable DR detector 107 along the imaging path 104 from position a to position c. Tomosynthesis projection images of the patient P are captured in the portable DR detector 107 at each angular imaging position of the tube head 101 as it is moved along the imaging path 104, which may be a linear path 104, or the imaging path 104 may be a curved path. Thus, capturing successive tomosynthesis projection x-ray images of a patient P at different relative angles may be achieved using a movable tube head 101 as described.

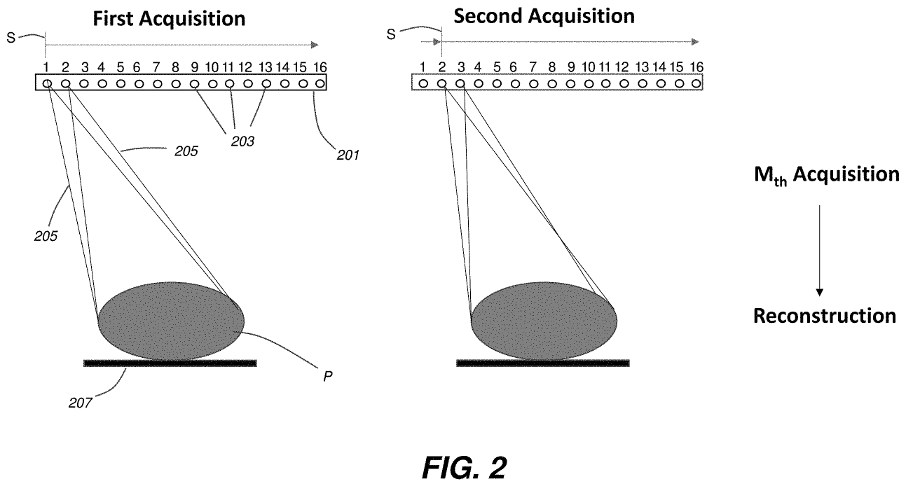

Turning to the schematic diagram of FIG. 2, there is illustrated a side view of a stationary tube head 201 having an array of x-ray sources 203 secured therein. The x-ray sources 203 may include carbon nanotube type x-ray sources, cold cathode type x-ray sources, or a combination thereof. The tube head 201 may be shaped as an elongated rectangular parallelepiped made from a substantially rigid radiopaque material having apertures formed in a bottom surface thereof each configured to shape and direct x-ray beams 205 emitted by a corresponding x-ray source 203 toward patient P and portable DR detector 207, which portable DR detector 207 is positioned behind patient P. In the example embodiment shown in FIG. 2 there are sixteen x-ray sources 203, numbered 1-16 left to right, disposed in the stationary tube head 201 with a corresponding aperture for each x-ray source 203 in a bottom surface of the tube head 201.

In one embodiment, the sixteen x-ray sources 203 may be controllably activated one at a time in a timed sequence starting at S with the x-ray source numbered 1 and continuing with each of x-ray sources 203 numbered 2-16, to acquire a series of sixteen radiographic images of patient P during one cardiac or respiratory cycle, shown in FIG. 2 as the First Acquisition. During a second cardiac or respiratory cycle acquisition, the numbered x-ray sources 203 may be activated in a wrap-around timed sequence by firing the x-ray sources 203 starting at S with the x-ray source 203 numbered 2 and continuing with each of the x-ray sources 203 numbered 3-16, then firing x-ray source #1, shown in FIG. 2 as the Second Acquisition. During a third cardiac or respiratory cycle acquisition, the numbered x-ray sources 203 may be activated in a wrap-around timed sequence starting with x-ray source 203 numbered 3, then continuing with sequential activation of x-ray sources numbered 4-16 and then sources numbered 1-2, and so on with each further acquisition M, wherein M is an integer. In general, during an M^{th} cardiac/respiratory cycle, patient images are acquired by firing the numbered x-ray sources 203 in a wrap-around sequence in the order M. M+1, M+2 and ending with M−1. After M acquisition sequences are performed, a first set of tomosynthesis images are selected by grouping the first image from each of the acquisition sequences, grouping the second image from each of the acquisition sequences, grouping the third image from each of the acquisition sequences, and so on, to form M sets, or groups, of acquired radiographic images. Each group, or set, so acquired will include images of the patient anatomy captured at a common phase in the periodic motion of the patient anatomy and each image in a group will be acquired at a different imaging angle, relative to the patient P and the DR detector 207. Each group so acquired may used to reconstruct a 3D radiographic image of the patient anatomy having less blurring caused by patient anatomy motion. Each reconstructed 3D image corresponds to a different phase of the patient anatomy motion cycle. Displaying the reconstructed 3D images in a timed sequence presents a 3D image in motion, i.e., 4D.

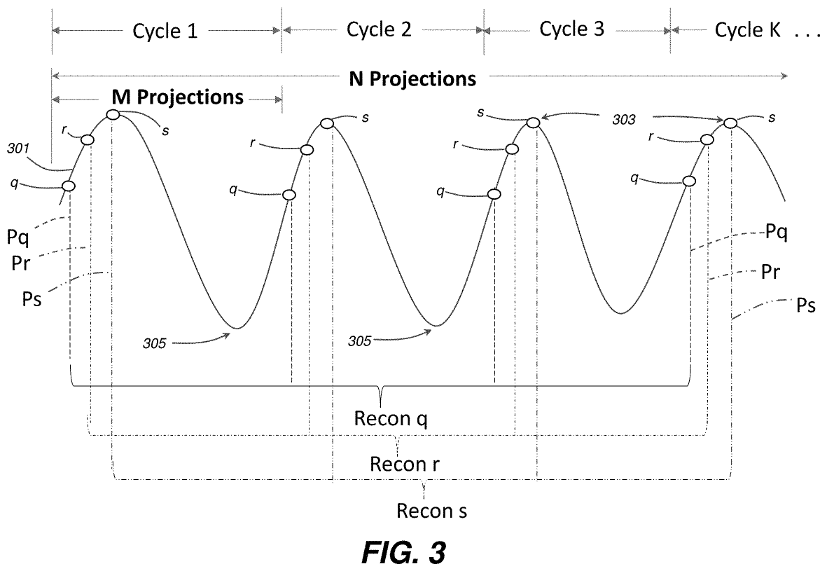

FIG. 3 is an illustration of periodic cycles 301, which may correspond to the expansion and contraction of cardiac or respiratory cycles in a patient. The peaks 303 of the cycles may represent full expansion of heart or lungs, and the troughs 305 of which may represent full contraction of heart or lungs. As described herein, a cardiac/respiratory cycle duration of any patient may be measured. Thus, the timing for firing x-ray sources in tube head 201 may be determined based on how many images per cycle of a patient P anatomy are desired to be captured. For example, if the first cycle, Cycle 1, duration is 1/60 s (e.g., single heart beat cycle) then the timing between each sequential firing of x-ray sources numbered 1-16 may be selected to be 1/960 s if all sixteen x-ray sources 203 are used. In another embodiment, if every other x-ray source 203 is used then the timing between each sequential firing of x-ray sources 203 numbered 1, 3, 5 . . . may be selected to be 1/480 s. As shown in FIG. 3, the timing for integer K example cycles are illustrated. The integer K instances at example time q on the periodic cycle 301 correspond to a same phase in each of the cycles 1-K; the integer K instances at example time r on the periodic cycle 301 correspond to a same phase in each of the cycles 1-K; and the integer K instances at example time s on the periodic cycle 301 correspond to a same phase in each of the cycles 1-K. Thus, q, r, s, each represent one of three different phases in each of cycles 1-K, wherein all the K radiographic images Pq captured at phase time q may be said to have a common phase, all the K radiographic images Pr captured at phase time r may be said to have a common phase different from the phase q radiographic images, and all the K radiographic images Ps captured at phase time s may be said to have a common phase different from the phase q and the phase r images. All the captured radiographic images may number N, an integer, including the combined images in groups Pq, Pr and Ps. The group of K images captured at phase q, Pq, may be used to reconstruct a 3D image of the patient anatomy at phase q, shown in FIG. 3 as Recon q; the group of K images captured at phase r, Pr, may be used to reconstruct a 3D image of the patient anatomy at phase r, shown in FIG. 3 as Recon r, and the group of K images captured at phase s. Ps, may be used to reconstruct a 3D image of the patient anatomy at phase s, shown in FIG. 3 as Recon s and so on. As described herein, sixteen (M) groups of images may each be used to reconstruct a 3D image if all sixteen x-ray sources 203 in the tube head 201 are used to capture radiographic images of patient P in each periodic cycle. It may be noted that any feasible number of x-ray sources 203 may be constructed in a tube head 201 and selectively activated to capture radiographic images of patient P as described herein.

As will be appreciated by one skilled in the art, aspects of the present invention may be embodied as a system, method, or computer program product. Accordingly, aspects of the present invention may take the form of an entirely hardware embodiment, an entirely software embodiment (including firmware, resident software, micro-code, etc.), or an embodiment combining software and hardware aspects that may all generally be referred to herein as a “service,” “circuit,” “circuitry,” “module,” and/or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Program code and/or executable instructions embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer (device), partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. Method comprising:
capturing a plurality of radiographic images of a portion of a patient, the portion of the patient in periodic motion;
selecting a first subset of the captured radiographic images, each image in the first subset having a common first capture time relative to a phase of the periodic motion; and
reconstructing a first 3D image using the first subset of captured radiographic images.

2. The method of claim 1, further comprising:
selecting a second subset of the captured radiographic images, each image in the second subset having a common second capture time relative to a phase of the periodic motion, the second capture time different from the first capture time; and
reconstructing a second 3D image using the second subset of captured radiographic images.

3. The method of claim 2, further comprising:
selecting a third subset of the captured radiographic images, each image in the third subset having a common third capture time relative to a phase of the periodic motion, the third capture time different from the second capture time; and
reconstructing a third 3D image using the third subset of captured radiographic images.

4. The method of claim 3, further comprising sequentially displaying in series the reconstructed first 3D image, the reconstructed second 3D image and the reconstructed third 3D image.

5. The method of claim 1, further comprising capturing the plurality of radiographic images of the portion of the patient each at a different acquisition angle.

6. The method of claim 1, wherein each image in the first subset having the common first capture time is captured at a different acquisition angle.

7. The method of claim 1, wherein the step of capturing comprises using a stationary x-ray source array having a plurality of x-ray sources distributed in a preselected pattern.

8. The method of claim 1, further comprising:
selecting additional subsets of the captured radiographic images, each additional subset having a common capture time relative to a phase of the periodic motion, the common capture time of each additional subset different from the common capture time of every other subset; and
reconstructing a 3D image for each of the additional subsets of captured radiographic images.

9. The method of claim 8, further comprising sequentially displaying in series the reconstructed first 3D image and the reconstructed 3D images for each of the additional subsets of captured radiographic images.

10. Method comprising:
capturing a plurality of radiographic heart or lung images of a patient, the heart or lungs of the patient in periodic motion;
selecting a first subset of the captured radiographic heart or lung images, each image in the first subset having a common first capture time relative to a phase of the periodic motion; and
reconstructing a first 3D image using the first subset of the captured radiographic heart or lung images.

11. The method of claim 10, further comprising:
selecting a second subset of the captured radiographic heart or lung images, each image in the second subset having a common second capture time relative to the phase of the periodic motion, the second capture time different from the first capture time; and
reconstructing a second 3D image using the second subset of the captured radiographic heart or lung images.

12. The method of claim 11, further comprising:
selecting a third subset of the captured radiographic heart or lung images, each image in the third subset having a common third capture time relative to a phase of the periodic motion, the third capture time different from the second capture time; and
reconstructing a third 3D image using the third subset of the captured radiographic heart or lung images.

13. The method of claim 12, further comprising sequentially displaying in series the reconstructed first 3D image, the reconstructed second 3D image and the reconstructed third 3D image.

14. The method of claim 10, further comprising capturing the plurality of radiographic heart or lung images each at a different acquisition angle.

15. The method of claim 10, wherein each image in the first subset having the common first capture time is captured at a different acquisition angle.

16. The method of claim 10, wherein the step of capturing comprises using a stationary x-ray source array having a plurality of x-ray sources distributed in a preselected pattern.

17. The method of claim 10, further comprising:
selecting additional subsets of the captured radiographic heart or lung images, each additional subset having a common capture time relative to the phase of the periodic motion, the common capture time of each additional subset different from the common capture time of every other subset; and
reconstructing a 3D image for each of the additional subsets of captured radiographic heart or lung images.

18. The method of claim 17, further comprising sequentially displaying in series the reconstructed first 3D image and the reconstructed 3D images for each of the additional subsets of captured radiographic heart or lung images.
